(19) United States
(12) Patent Application Publication
(10) Pub. No.: US 2014/0114396 A1
(43) Pub. Date: Apr. 24, 2014

(54) COVERED STENT FOR VASCULAR CLOSURE

(71) Applicant: Boston Scientific Scimed, Inc., Maple Grove, MN (US)

(72) Inventors: Roger Hastings, Maple Grove, MN (US); Jason Hill, Brooklyn Park, MN (US)

(73) Assignee: Boston Scientific Scimed, Inc., Maple Grove, MN (US)

(21) Appl. No.: 14/139,162

(22) Filed: Dec. 23, 2013

Related U.S. Application Data
(63) Continuation of application No. 12/982,080, filed on Dec. 30, 2010.
(60) Provisional application No. 61/291,234, filed on Dec. 30, 2009.

(51) Int. Cl. A61F 2/90

(52) U.S. Cl. CPC A61F 2/90
USPC 623/1.15

(57) ABSTRACT

A stent assembly includes a stent, a covering on at least a portion of the stent, and a string encircling at least a portion of the covering. The string is releasably engaged to the covering or stent or both the covering and the stent. The string can be adhered to the stent, or the stent assembly, to the covering of the stent assembly, or both the covering and the stent. The string can be wrapped around the stent or covering in an interwoven loop or knit pattern. The covering can be made to overlap a perforation in a body lumen such as an artery or blood vessel and prevent bleeding.

### CROSS-REFERENCE TO RELATED APPLICATIONS

This Application is a continuation of U.S. application Ser. No. 12/982,080, filed Dec. 30, 2010, which is a non-provisional of U.S. Application No. 61/291,234, filed Dec. 30, 2009, the entire contents of which are herein incorporated by reference.

### STATEMENT REGARDING FEDERALLY SPONSORED RESEARCH

Not Applicable

### BACKGROUND OF THE INVENTION

In some embodiments this disclosure relates to implantable medical devices, their manufacture, and methods of use. Some embodiments are directed to delivery systems which are utilized in the delivery of such devices.

### Description of the Related Art

Stents and similar devices such as stent-grafts, expandable frameworks, and similar implantable medical devices, are radially expandable endoprostheses which are typically intravascular implants capable of being implanted transluminally and enlarged radially after being introduced percutaneously.

Stents are often utilized or combined with grafts (e.g., stent-grafts). Grafts typically are made from textile or non-textile materials that are woven together to from a tubular structure through which blood or other bodily fluids can flow. Grafts can be configured to permit the ingrowth of cells in order to promote stability of the implanted graft within a body lumen.

An example of a stent-graft is shown in U.S. Pat. No. 6,626,939 to Burnside et al., thereby incorporating benefits of both the stent and graft. Stent-grafts may be implanted in a variety of body lumens or vessels such as within the vascular system, urinary tracts, bile ducts, fallopian tubes, coronary vessels, secondary vessels, etc.

All U.S. patents and applications and all other published documents mentioned anywhere in this application are incorporated herein by reference in their entirety.

Without limiting the scope of this disclosure, a brief summary of some of the claimed embodiments is set forth below. Additional details of the summarized embodiments may be found in the Detailed Description of the Invention below.

A brief abstract of the technical disclosure in the specification is provided for the purposes of complying with 37 C.F.R. §1.72.

### BRIEF SUMMARY

In at least one embodiment, this disclosure is directed to a stent assembly comprising a stent having an unexpanded configuration and an expanded configuration and a first string having a portion thereof formed in a repeating knit pattern. The repeating knit pattern portion of the first string encircles at least a portion of the stent in the unexpanded configuration.

### BRIEF DESCRIPTION OF THE SEVERAL VIEWS OF THE DRAWINGS

A detailed description of exemplary embodiments are hereafter described with specific reference being made to the drawings.

FIG. 1A is a perspective view of an embodiment of a stent assembly.

FIG. 1B shows the embodiment of FIG. 1A in an unexpanded configuration.

FIG. 2 is a perspective view of a portion of a stent of FIG. 1A.

FIG. 3A is a perspective view of an embodiment of the stent assembly, wherein a covering is disposed circumferentially around a portion of the stent.

FIG. 3B is a perspective view of an embodiment of the stent assembly, wherein the covering is disposed longitudinally in a strip about a portion of the stent.

FIG. 4 is a perspective view of an embodiment of the stent assembly, wherein the covering is disposed within a portion of the stent.

FIG. 5A is a cross sectional view of an embodiment of the stent assembly.

FIG. 5B is a cross sectional view of an embodiment of the stent assembly.

FIG. 6A is a side view of an embodiment of the stent assembly within a body lumen.

FIG. 6B is a side view of the embodiment of FIG. 6A within a body lumen.

FIG. 6C is a side view of the embodiment of FIGS. 6A and 6B within a body lumen.

FIG. 7 is a perspective view of an embodiment of the stent assembly.

FIG. 8A is a side view of an embodiment of the stent assembly.

FIG. 8B is a detailed side view of the embodiment of FIG. 8A.

FIG. 9A is a side view of an embodiment of the stent assembly.

FIG. 9B is a detailed side view of the embodiment of FIG. 9A.

FIG. 10 is a side view of an embodiment of the stent assembly.

FIG. 11A is a side view of an embodiment of the stent assembly within a body lumen.

FIG. 11B is a side view of the embodiment of FIG. 11A of the stent assembly within a body lumen.

FIG. 11C is a side view of the embodiment of FIGS. 11A and 11B of the stent assembly within a body lumen.

FIG. 12A is a side view of an embodiment of the stent assembly being inserted into a body lumen.

FIG. 12B is a side view of the embodiment of FIG. 12A of the stent assembly being inserted into a body lumen.

FIG. 12C is a side view of the embodiment of FIGS. 12A and 12C of the stent assembly being inserted into a body lumen after deployment.

FIG. 13 is a side view of an embodiment of the stent assembly.

### DETAILED DESCRIPTION OF THE INVENTION

For the purposes of this disclosure, like reference numerals in the figures shall refer to like features unless otherwise indicated.

In some embodiments, a stent assembly is provided for trans-luminal delivery into a body cavity, for example an artery, blood vessel, vein, or other body lumen. In at least one embodiment, the stent assembly comprises a stent and at least one covering adjacent to at least a portion of the stent.

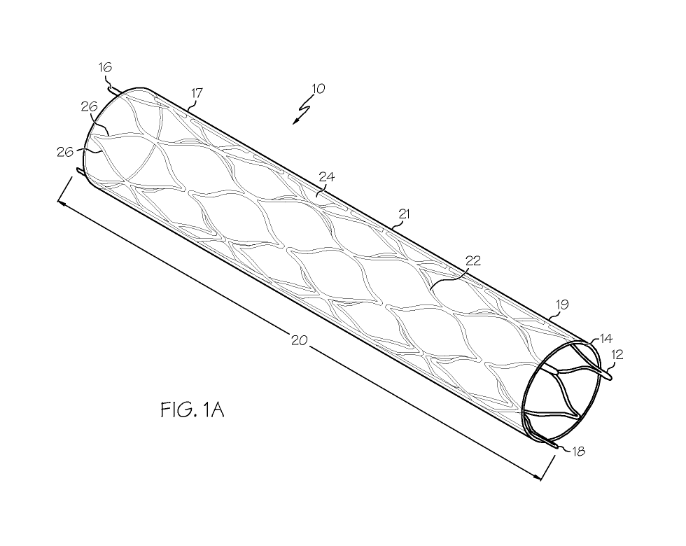

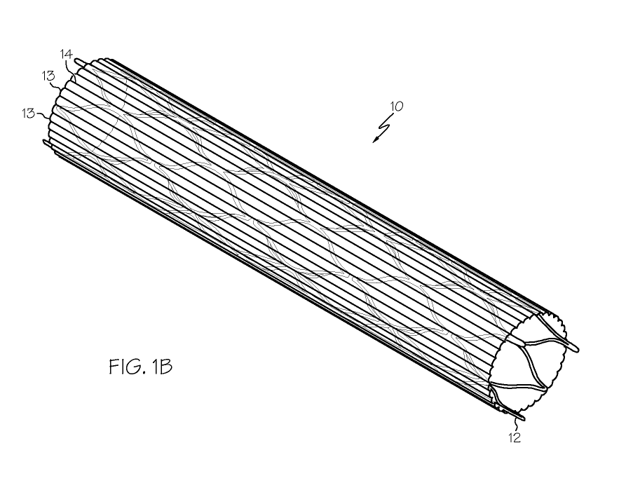

As shown in FIG. A1, a stent assembly 10 comprises a stent 12 and a covering 14 encircling at least a portion of the stent 12. The stent assembly 10 of FIG. 1A is shown in an expanded configuration. In some embodiments, the stent 12 of the stent assembly 10 comprises a self expanding stent.

The stent 12 is shown with a proximal end 16, a distal end 18, and a length 20 between the proximal and distal ends. Furthermore, the stent has a proximal portion 17, a distal portion 19, and a middle 21 therebetween. The stent 12 defines a stent outer surface 32 and a stent inner surface 34 (FIG. 3). In some embodiments, the stent 12 has a framework 22 defining a plurality of open cells 24. In some embodiments, each cell 24 has an area of less than approximately 5 sq. mm, when the stent 12 is in an expanded configuration.

In some embodiments, for example as shown in FIG. 1B, the covering 14 comprises a plurality of folds 13 when the stent 12 is in an unexpanded configuration. Thus, when the stent 12 is expanded, the covering 14 is permitted to expand in conjunction with the stent 12.

In some embodiments, the stent assembly 10 comprises an assembly outer surface 38 and an assembly inner surface 39 (FIG. 3). The assembly outer surface 38 is defined as the outermost surface of stent assembly 10 for any given region of the stent assembly. For example, where the stent assembly 10 includes only a stent and a single covering 14 disposed exteriorly to a portion of the stent 12, the term “assembly outer surface” refers to the outer surface of the covering 14 for the portion(s) of the assembly where the covering is located exteriorly to the stent 12. Moreover, and for the purposes of illustration, the portions of the stent 12 that do not have covering 14 disposed exteriorly to that particular portion of the stent 12, the “assembly outer surface” properly refers to the outer surface of the stent 12 for the portions of the stent lacking the aforementioned covering 14. Furthermore, in an instance where the stent assembly 10 includes a covering 14 disposed exteriorly to a portion of the stent 12 and the covering 14 has a bioactive agent disposed exteriorly to the covering 14, the term “assembly outer surface” refers to the outer surface of the bioactive agent which is disposed exteriorly over the covering. Moreover, in an instance where the bioactive agent is disposed exteriorly over the covering 14, and the covering 14 is disposed exteriorly over only a portion of the stent 12, the outer surface of the portion of the stent 12 without the covering 14 and bioactive agent is properly referred to as the “assembly outer surface” for that portion of the stent assembly.

The term “assembly inner surface” 39 is defined in a fashion similar to the assembly outer surface, discussed above. In short, the assembly inner surface refers to the innermost surface of the stent assembly 10 for a given region of the stent assembly.

A variety of stent or stent-graft designs can be used with some or all of the embodiments of the present invention. Suitable stents include, but are not limited to stents shown and described in U.S. Pat. Nos. 4,954,126; 6,139,573; 7,037,330; 7,122,059; 7,223,283; and 7,381,217; all of which are incorporated herein by reference, may be suitable. In some embodiments, stents with relatively narrow, thin struts or filaments and an open stent architecture are used. In this way, the stent is permitted to hold open the covering upon expansion of the stent assembly. In addition, in some embodiments, blood or fluid pressure can aid in expanding the covering and stent assembly.

In some embodiments, the framework 22 is comprised of a self-expanding metal, for example a NiTi alloy. Other suitable materials include, but are not limited to Elgiloy® or plastic. The framework 22 can also be biodegradable, for example a biodegradable plastic such as poly(lactic-co-glycolic) acid (PLGA), polylactic acid (PLA), or polyglycolic acid (PGA). The above listed materials are meant to be non-limiting. Other suitable materials can also be used.

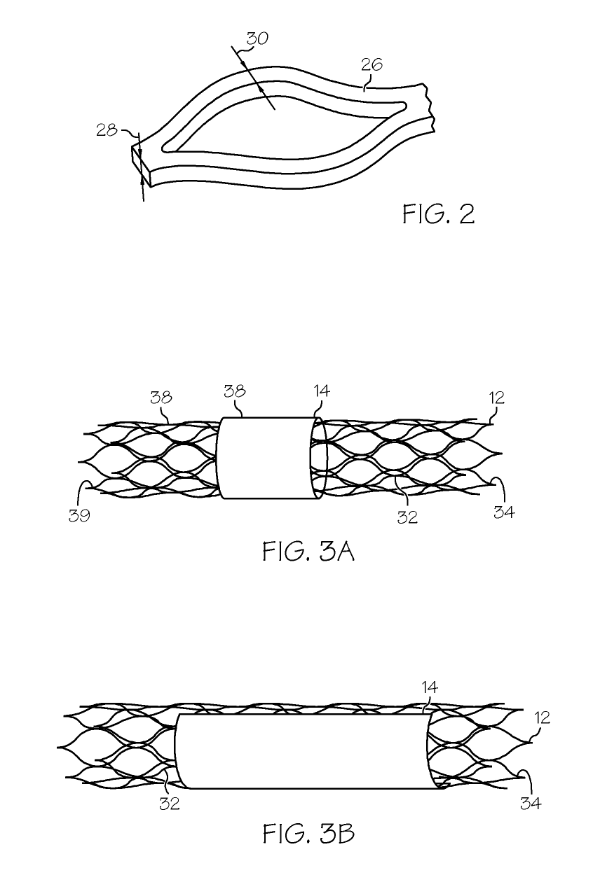

The stent framework 22 can comprise a plurality of struts 26, which, in some embodiments, have a thickness 28 of 0.0008 in.-0.004 in. In some embodiments, the struts have a thickness 28 of 0.003 in. The struts 26 also have a width 30, measured perpendicularly to the longitudinal direction of the strut, as shown in FIG. 2. The width 30 can also be in the range of 0.0008 in.-0.004 in. A corresponding strut cross section can therefore be square or rectangular. Other suitable shapes, such as circular or elliptical are also permitted. In some embodiments, the stent 12 has an expanded length to diameter ratio of between approximately 1:1 and 3:1. For example, where the stent or stent assembly is introduced using a delivery system designed to prevent tilting or misalignment of the stent during introduction, the length to diameter ratio can be less than without use of such a delivery system. Where such a delivery system is employed, the length to diameter ratio can be approximately 1:1. In some embodiments, the stent diameter can be approximately 4-30 mm, and in some embodiments is 5-8 mm. In some embodiments, the thickness of the covering 14a (or 14b, or 14c) is less than 0.002 in. In some embodiments, the thickness of the covering 14a (or 14b, or 14c) is 0.001 in. In some embodiments, the overall thickness 29 (FIG. 5A) of the stent assembly 10 ranges from less than 0.0018 to approximately 0.010 in. In some embodiments, the overall thickness 29 of the stent assembly is 0.005 in.

In some embodiments, a covering 14 is adjacent to the stent outer surface 32. The covering 14 can be adjacent to the entirety of the stent outer surface 32 (FIG. 1), or a portion of the stent outer surface 32. For example, the covering 14 can extend circumferentially around a portion of the stent 12, as shown in FIG. 3A, longitudinally along a portion of the stent 12, for example as shown in FIG. 3B, or in any other suitable manner.

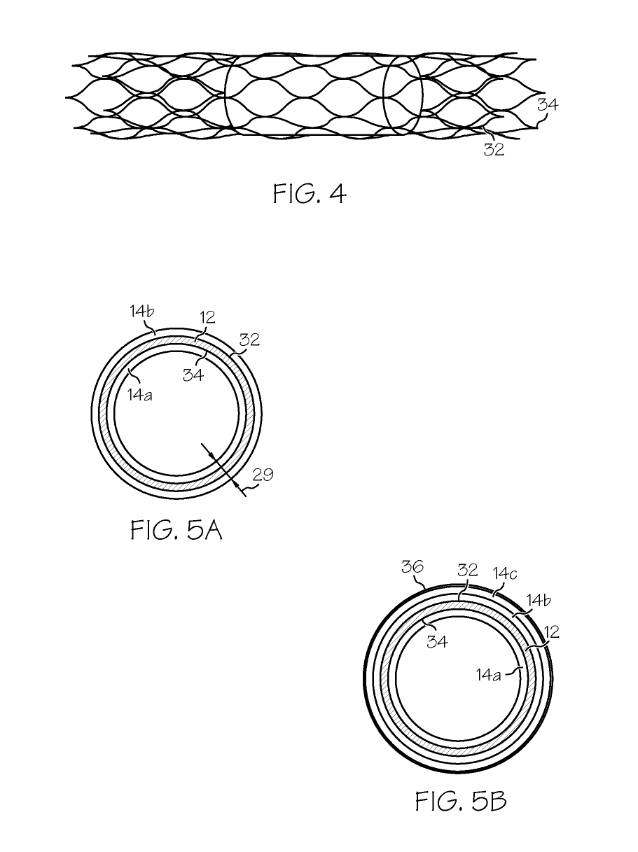

In some embodiments, the covering 14 is adjacent to the stent inner surface 34. Stated differently, the stent encircles the covering 14. The covering 14 can be adjacent the entirety of the stent inner surface 34, or a portion of the stent inner surface 34. In addition, the covering 14 can extend in an annular fashion within a portion of the stent inner surface 34, for example as shown in FIG. 4, longitudinally along a portion of the stent inner surface 34, or in any other suitable configuration.

In some embodiments, the covering 14 can be immediately adjacent to the stent inner or outer surfaces 32, 34. In this way there can be no material or layer between the specified surface of the stent (inner or outer) and the covering 14.

As shown in FIG. 5A, in at least some embodiments the stent 12 comprises a first layer of covering (or first covering) 14a on the stent inner surface 34 and a second layer of covering (or second covering) 14b on the stent outer surface 32 of the stent. In addition, the stent can have a plurality of coverings 14a, 14b, 14c (FIG. 5B) located on the inner or outer surfaces or both the inner and outer surfaces 34, 32. The coverings 14a, 14b, 14c can comprise the same material or different materials.

In some embodiments, for example where the stent 12 has a first layer of covering 14a and a second layer of covering 14b, the first and second layers are thermally combined to encapsulate the stent framework 22 within the thermally combined material. For example, where the first and second layers of covering 14a, 14b comprise ePTFE, the layers can be thermally attached to each other, thereby forming microscopic mechanical connections between the first and second layers 14a, 14b, or portions thereof. Where the first and second layers are thermally combined, they cannot be separated without damage to one or both of the layers. Thermally combining first and second layers has the advantage of providing increased adherence to the framework 22 when compared to uncombined first and second layers.

In some embodiments, the covering 14 comprises a composite material, for example having a microporous structure. In some embodiments, the covering 14 comprises a microporous polymer, for example expanded polytetrafluoroethylene (ePTFE), which in some embodiments is combined with an elastomeric material, for example polycarbonate urethane, polystyrene-polyisobutylene-polystyrene triblock copolymer (SIBS), or silicone. The covering 14 can also comprise nonporous elastomers. In some embodiments, the covering comprises pores or micro-structures that facilitate in-growth of cells and promote healing of the affected body cavity or lumen.

In some embodiments, a first layer of covering 14a can be adhered to a second layer of covering 14b with an elastomer. In some embodiments, the first and second layers 14a, 14b are ePTFE.

In some embodiments, the covering 14 has a coating 36 disposed thereon. In some embodiments, the coating 36 comprises a therapeutic agent. At least a portion of the stent can be configured to include one or more mechanisms for the delivery of the therapeutic agent. For example, the therapeutic agent will be in the form of a layer (or layers) of material placed on a surface region of the stent 12 or covering 14, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

Desirably, the covering 14 comprises a material that is capable of self-sealing, for example to permit re-entry into an artery or vessel. Elastomeric materials such as silicone are well suited to permit sealing of the covering after re-entry. As used herein, the term “elastomeric” describes materials that have long chain molecules which are capable of recovering to an original shape after being stretched, so as to resist plastic deformation. Further “elastomeric” refers to materials capable of extending from 5-700% without undergoing plastic deformation. Examples of suitable elastomeric materials are: silicone, polyurethane, poly(styrene-isobutylene-styrene) block copolymer, and polycarbonate urethane.

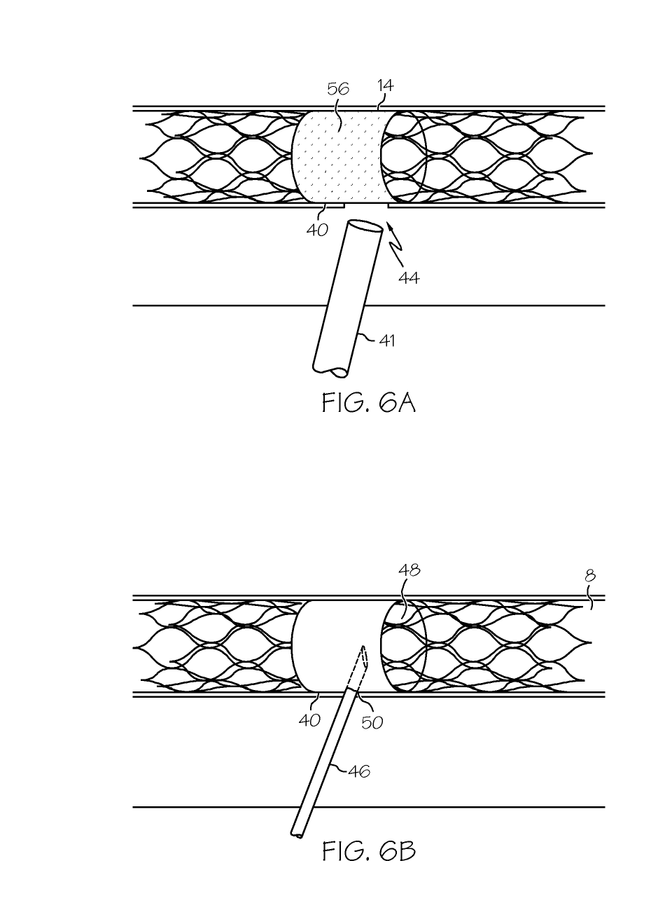

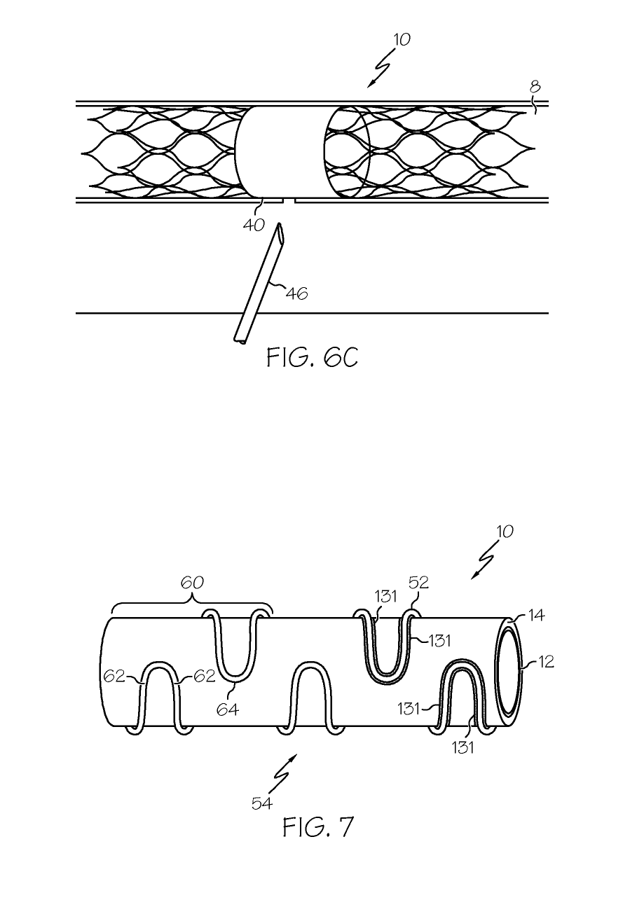

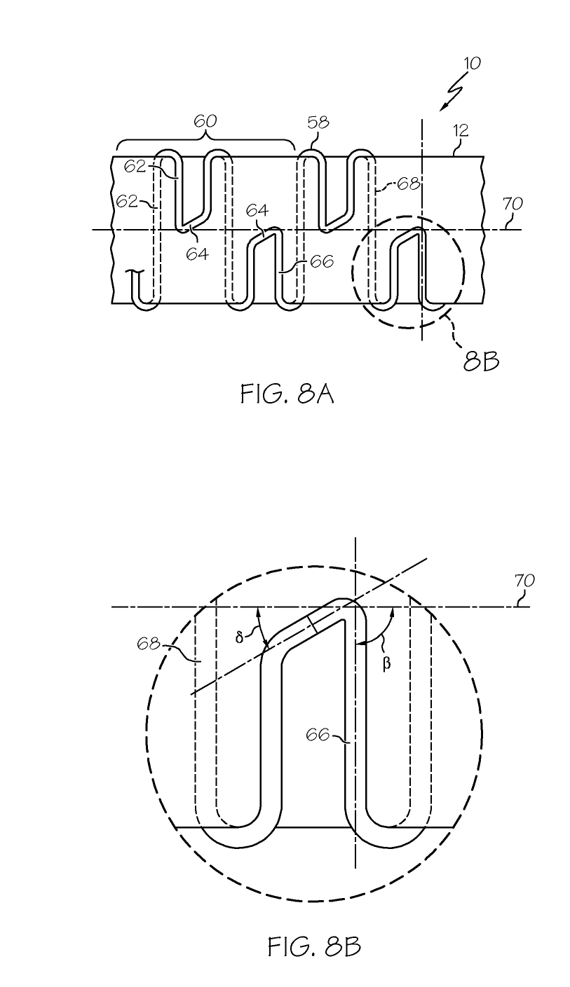

In some embodiments, the covering 14 can be comprised of a non-porous material, for example where the length to diameter ratio of the stent or stent assembly is less than approximately 1:1. In addition, the covering 14 may be non-porous, for example where the covering is isolated to an area near the arteriotomy. The covering 14 can also comprise a semi-porous material, for example: fibrous silicone, polyurethane, poly(ethylene terephthalate), poly(styrene-isobutylene-styrene) block copolymer, and polycarbonate urethane. In some embodiments, the porosity is defined by a 30-75% void volume with a pore size distribution between 0.1 and 10 microns. The semi-porous material can be impregnated with a bio-absorbable material such as starch powder or poly(ethylene glycol) (PEG), for example, to promote sealing of the semi-porous material. A bio-absorbable material (e.g., starch powder) can also be placed in the vicinity of the arteriotomy to assist in sealing. As shown in FIG. 6A, an embodiment of the stent assembly is shown after deployment in a body lumen. The covering 14 comprising a self-sealing material 40 is shown overlaying a perforation 44, created by an introducer 41 or other medical instrument prior to insertion of the stent assembly 10.

In some embodiments, the stent assembly 10 can further comprise a tissue adhesive 56, for example as shown in FIG. 6A. In some embodiments, the tissue adhesive 56 provides an additional measure of protection to ensure sealing of the perforation. Suitable tissue adhesives can be found, for example, in US Publication No. 2009/0044895, which is herein incorporated by reference. In some embodiments, the tissue adhesive 56 can be placed on the covering 14, or a portion thereof. In some embodiments, the tissue adhesive 56 can be disposed in the covering 14 or a portion thereof, for example where the covering 14 comprises a porous material. In this way, the tissue adhesive 56 is permitted to exude out of the covering 14. As the covering 14 is pressed up against the artery or lumen wall, the tissue adhesive 56 flows out of the covering and onto the adjacent tissue surface, thereby sealing the puncture. The tissue adhesive 56 can be placed in pores or holes in the covering, for example, where the tissue adhesive is susceptible to reacting with blood or other fluid within the body lumen. Thus, exposure of the tissue adhesive 56 is minimized prior to deployment of the stent and covering. Material bonding sites of the tissue adhesive are therefore not consumed by reaction with blood (or other fluid). Moreover, the tissue adhesive 56 can have a cure time that is longer than the time necessary to effectuate stent deployment, thus ensuring that the stent is deployed before the tissue adhesive 56 can no longer bond to the adjacent tissue.

FIG. 6B shows the stent assembly of FIG. 6A during a re-entry procedure into a body lumen 8, for example a femoral artery. The self-sealing material 40 is pierced by the instrument 46, thereby permitting access to the interior of the stent assembly 48 via hole 50 in the covering 14. The instrument 46 is inserted through an open cell 24 in the stent framework 22.

Upon removal of the instrument 46, as shown in FIG. 6C, the self-sealing material 40 rebounds to close the hole 50. Thus, the stent assembly 10 and body lumen 8 can be re-accessed subsequent to initial deployment of the stent assembly 10 and, after the re-access procedure is complete the self-sealing material 40 re-seals. The self-sealing material 40 thereby minimizes the need to wait for clotting, and the patient should be ambulatory immediately, with no need to hold pressure on the access site. Another advantage of the self-sealing material 40 is that it permits self-sealing after one or more subsequent piercings. In at least one embodiment, fluid pressure from the body lumen 8 aids in re-sealing.

As shown in FIG. 7, some embodiments of the stent assembly 10 comprise a filament or string 52 or a plurality of strings 52 encircling at least a portion of the stent 12. In some embodiments, the string(s) 52 retain the stent 12 in an unexpanded configuration prior to deployment.

As used herein, the term “string” means a flexible cord or filament having a non-hollow cross-section and an overall length, the non-hollow cross-section being small when compared to the overall length; the string may be formed of a single strand or a plurality of strands which can be braided together or otherwise interlaced; the string is defined by a first end and a second end and the overall length of the string is defined as the distance between the first end and the second end. The non-hollow cross section can be circular, in which case the diameter of the string is small when compared to the overall length. The non-hollow cross section can also be elliptical, flat, like a ribbon, or any other suitable non-hollow shape.

In some embodiments, the string 52 is wrapped or looped around a portion of the stent 12 when the stent is in an unexpanded configuration. The string 52 can also be wrapped around a portion of the covering 14. In some embodiments, the string 52 is adhered to the stent 12 or covering 14 in a serpentine path 54, for example as shown in FIGS. 7, 8. In some embodiments, the string is adhered to the stent 12 or covering 14 of the stent assembly with an adhering material 131. In some embodiments, the string is knit and/or looped around a portion of the stent 12 and adhered to the stent 12 or covering 14. In some embodiments, at least a portion of the string 52 defines a repeating pattern. The repeating pattern can comprise a woven, braided, knitted, looped, wrapped, laced, or otherwise suitable pattern.

In some embodiments, the string 52 comprises a series of sequential wraps 60. The sequential wraps 60 comprise portions which extend in a circumferential direction and portions which extend in a longitudinal direction. In some embodiments, the sequential wraps 60 can comprise portions extending both circumferentially and longitudinally around a portion of the stent. Further, in some embodiments, the sequential wraps 60 comprise straight portions and end portions. The sequential wraps 60 can comprise additional shapes, for example sinusoidal, curvilinear, or any other suitable shape.

As shown in FIG. 7 the stent comprises straight portions 62, and end portions 64. The end portions 64 can comprise a curve, semi-circle, U-shape, V-shape, or any other suitable shape.

Turning to FIG. 8A, in some embodiments, the straight portions 62 are parallel to one another. The straight portions 62 can be perpendicular to the longitudinal axis 70 of the stent assembly 10, or the straight portions 62 can extend at an angle β relative to the longitudinal axis 70 of the stent assembly 10.

In some embodiments, the end portions 64 are parallel to one another and parallel to the longitudinal axis of the stent assembly 10. In some embodiments, the end portions 64 extend at an angle δ relative to the longitudinal axis 70 of the stent assembly 10.

In some embodiments, the straight portions 62 comprise a long straight portion 66, and a short straight portion 68, for example as shown in FIG. 8A. With further reference to FIG. 8A, a single sequential wrap 60 comprises one complete cycle; as shown, a single sequential wrap includes an end portion 64 followed by a short straight portion 68 followed by another end portion 64 followed by a long straight portion 66. The sequential wrap 60 can also include straight portions 62 that are all the same length as one another, for example as shown in FIG. 7.

As shown in FIG. 8A, the end portions are parallel to one another. However, the end portions 64 can also be non-parallel, or perpendicular to one another. The straight portions 62 and end portions 64 can also comprise any other suitable configuration.

Furthermore, a portion of the end portions 64, shown in FIG. 8A, overlap the longitudinal axis 70 of the stent 12, or a projection of the longitudinal axis 70. The end portions 64 are shown as being longitudinally offset from one another along the length of the stent. In addition, portions of the end portions 64 are shown overlapping each other circumferentially around the perimeter of the stent 12 or covering 14. The end potions 64 need not overlap, however, and the string 52 can extend around only a portion of the circumference of the stent 12 or covering 14.

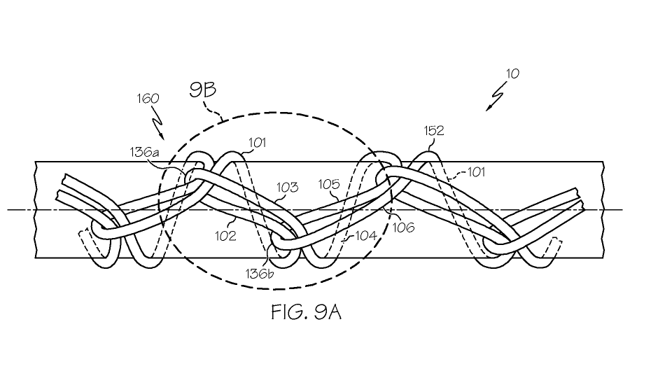

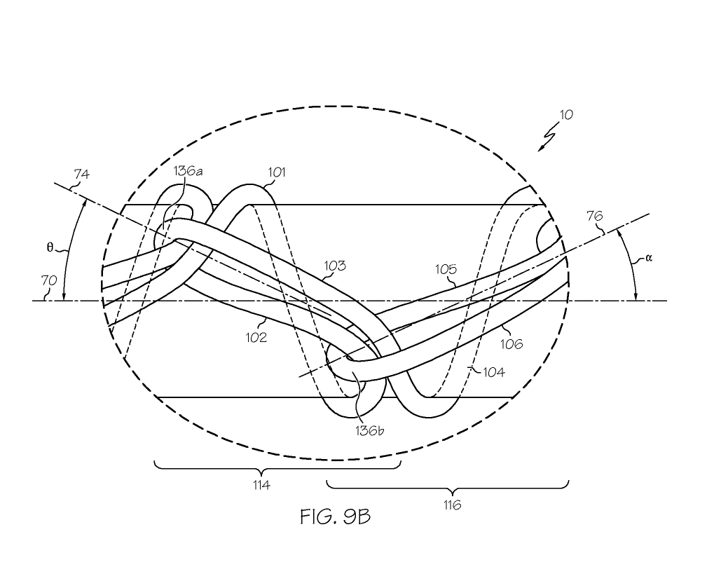

As shown in FIG. 9A, some embodiments comprise a string 152 that encircles the stent 12 or stent covering 14 in a knit pattern 160. In some embodiments, the knit pattern can comprise an interwoven loop pattern, for example as shown. The string 152 in the knit pattern 160 comprises head ends 136a, 136b of the knit. The string of the knit pattern 160 comprises a plurality of string sections, including a first string section 101, a second string section 102, a third string section 103, a fourth string section 104, a fifth string section 105, and a sixth string section 106.

As further shown in FIG. 9A, the first string section 101 flows from the sixth string section 106; the second string section 102 flows from the first string section 101; the third string section 103 flows from the second string section 102; the fourth string section 104 flows from the third string section 103; the fifth string section 105 flows from the fourth string section 104; and the sixth string section 106 flows from the fifth string section 105; at this point the pattern repeats itself.

Portions of the string sections (e.g., a first string section 101, second string section 102, third string section 103, fourth string section 104, fifth string section 105, sixth string section 106) overlap one another as shown in FIG. 9A. The points of overlap define the place at which one string section (101, 102, 103, 104, 105, 106) flows into the next string section. For example, string section 101 flows into string section 102 where string section 101 is overlapped by string section 105.

Turning to FIG. 9B, in some embodiments, knit pattern 160 comprises a series of segments including a first segment 114 and a second segment 116, the first segment 114 having a first head end 136a and the second segment having a second head end 136b. The first segment 114 comprises string sections 101, 102 and 103, while the second segment 116 comprises string sections 104, 105 and 106. Thus, in some embodiments, the knit pattern repeats as first segment 114, second segment 116, first segment 114, second segment 116, and so forth. As shown in FIG. 9B, the first and second segments 114, 116 overlap each other along a portion of the segments, for example at the first and second head ends 136a, 136b.

In some embodiments, the first head end 136a has a longitudinal axis 74, which bisects the string sections 102 and 103, as shown in FIG. 9B. The longitudinal axis 74 of the first head end 136a forms an angle θ relative to the longitudinal axis 70 of the stent assembly 10.

The second head end 136b has a longitudinal axis 76, which bisects the string sections 105 and 106. The longitudinal axis 76 of the second head end 136b forms and angle α relative to the longitudinal axis 70 of the stent assembly 10. In some embodiments, angle α is equal to angle θ. That is:

α=θ

In some embodiments, the first string section 101 wraps around a portion of the covering 14 and extends from the overlap of string section 103 to the overlap of string section 106. String section 102 extends from the overlap of string section 106 to the overlap of string section 101. String section 103 extends from the overlap of string section 101 to the overlap of string section 105. String section 104 extends from the overlap of string section 106 to the overlap of string section 103 of the second loop. String section 105 extends from the overlap of string section 103 of the second loop to the overlap of string section 102 of the first loop. String section 106 extends from the overlap of string section 102 of the first loop to the overlap of string section 103 of the second loop.

In some embodiments, a portion of string 52, 152 is adhered to the stent 12 or covering 14 with an adhering material 131 (FIG. 7). Although shown on only a portion of two sequential wraps 60 of the string 52, the adhering material 131 can be used to adhere all of the wraps 60, portions of all of the wraps 60, or portions of only some of the wraps 60. In addition, the adhering material 131 can be disposed on the covering and/or stent, or portions of either the covering or the stent or both the covering and the stent, for example where the coving encircles only a portion of the stent (e.g., FIGS. 3A and 3B). The adhering material 131 can further be employed in any other suitable configuration.

Suitable materials for adhering the knit pattern 160 or sequential wraps 60 to the stent 12 or covering 14 include, but are not limited to sugars such as sucrose, maltose, or dextrose, for example where the stent assembly is fully bio-absorbable. In at least one embodiment, the stent 12 can comprise PLGA, the cover 14 can comprise PGA, and the knit pattern 160 is adhered to the covering 14 via sucrose, maltose, or dextrose. Alternatively, for example where the stent assembly 10 is intended to be non bio-absorbable, the covering 14 can comprise an elastomer such as silicone, polyurethane, SIBS, or ePTFE. In some embodiments, the stent 12 of the stent assembly 10 comprises NiTi and the covering 14 comprises ePTFE.

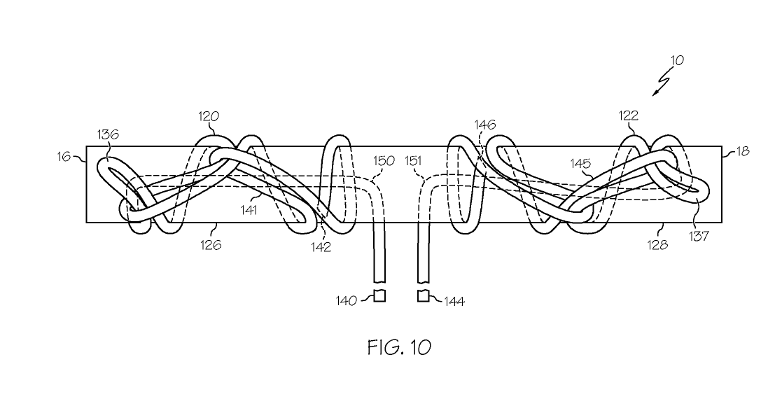

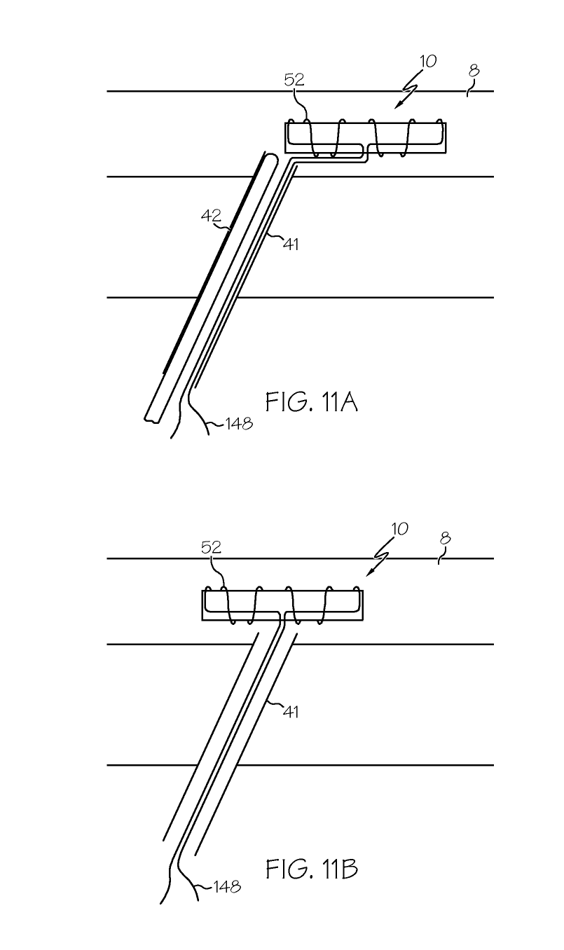

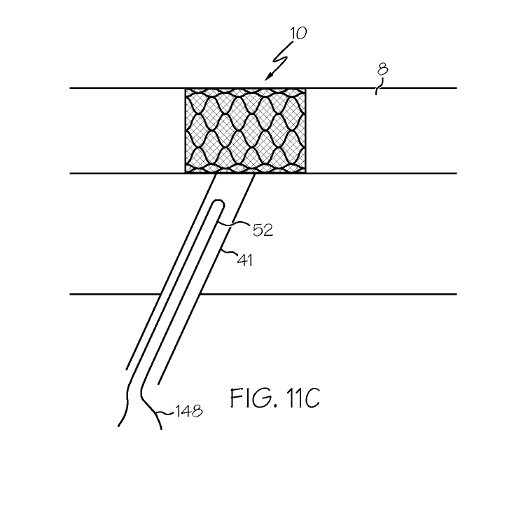

In some embodiments, for example where the stent assembly 10 comprises two strings, a first string 120 and a second string 122, the first and second strings 120, 122 can encircle at least a portion of the stent 12 or covering 14, as shown in FIG. 10. In some embodiments, the first string 120 encircles a proximal portion 126 of the stent or covering, and the second string 122 encircles a distal portion 128 of the stent or covering. The first string 120 can have head ends 136 pointing toward the proximal end 16 of the stent. The second string 122 can have head ends 137 pointing toward the distal end 18 of the stent. In some embodiments, the first head ends 136 can alternatively point toward the distal end 18 of the stent 12 and the second head ends 137 can point toward the proximal end 16 of the stent 12.

The first string 120 comprises a first end 140, a second end 142 and a middle region 141 therebetween. Similarly, the second string 122 comprises a first end 144, a second end 146 and a middle region 145 therebetween. In some embodiments, for example where the stent assembly 10 comprises two strings, including a first string 120 and a second string 122, the first ends 140, 144 of the first and second strings 120, 122 are not adhered to the stent 12 or covering 14. In some embodiments, the second ends 142, 146 of the first and second strings 120, 122 are adhered to the stent 12 or covering 14. In some embodiments, the second ends 142, 146 can be wrapped under an adjacent string section to frictionally hold the second ends 142, 146 to the stent 12 or covering 14 beneath the adjacent string section prior to release, for example as shown in FIG. 10. Thus, the attached ends can alternatively be referred to as “secured ends” and the non-attached ends can alternatively be referred to as “free” ends.

In some embodiments, the first string 120 can comprise a bend 150, and the second string 122 can comprise a bend 151. The first string 120 can be adhered to the stent 12 or covering 14, for example along the length of the string 120 from the second end 142 to the bend 150. Similarly, the second string 122 can be adhered to the stent 12 or covering 14 along the length of the string 122 from the second end 146 to the bend 151.

In some embodiments, only portions of the string or strings are adhered to the stent 12 or covering 14. However, adhering material (not shown) desirably extends up to the bends 150, 151 in order to properly position the stent assembly within a body lumen, as is discussed in greater detail below.

In a manner similar to that described above with respect to the stent assembly 10 having a knit pattern, the stent assembly 10 having sequential wraps, for example as shown in FIG. 8A, can comprise a plurality of strings.

Where the stent assembly 10 comprises only a single string, both ends can be free ends 148, and the string 52, 152 can be secured to the stent 12 or covering 14 along the middle region of the string.

In some embodiments, the stent or stent assembly is compressed into an unexpanded configuration. Subsequently, the string(s) are wrapped or adhered to the stent or stent assembly to hold the same in an unexpanded configuration. Upon implantation and removal of the string(s), the stent or stent assembly is permitted to self-expand to an expanded configuration.

Turning to FIG. 11A, insertion and operation of the stent assembly is described below with reference to stent assembly 10. However, the described method of insertion is applicable to any and all of the embodiments and variations employing string deployment. In addition, it bears noting that the insertion method described below is performed without the use of a catheter and the stent assembly is in an unexpanded configuration prior to delivery.

In some embodiments, the stent assembly 10 is inserted into the body lumen via an introducer sheath 41 and the stent 12 (or stent-graft) is subsequently positioned within the body lumen 8, for example with a push rod 42. A free end or ends 148 of the string 52 remains outside of the patient's body and is accessible to the operator. To position the stent assembly 10 within the body cavity (e.g., lumen), the free end 148 of the string 52 is pulled by the operator, as shown in FIG. 11B.

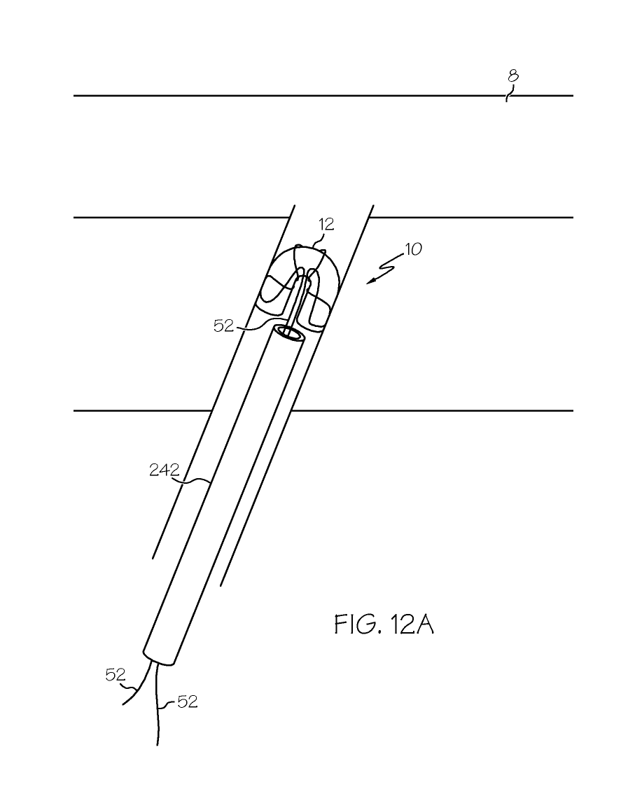

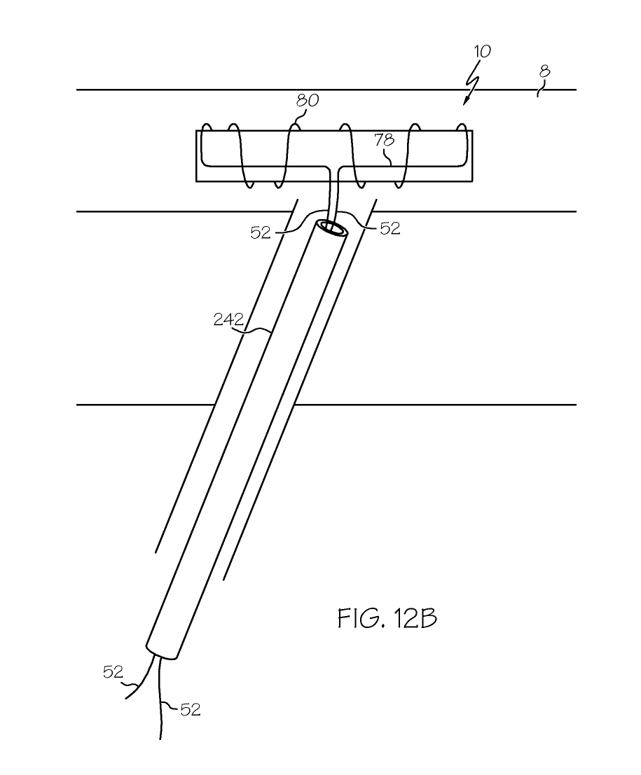

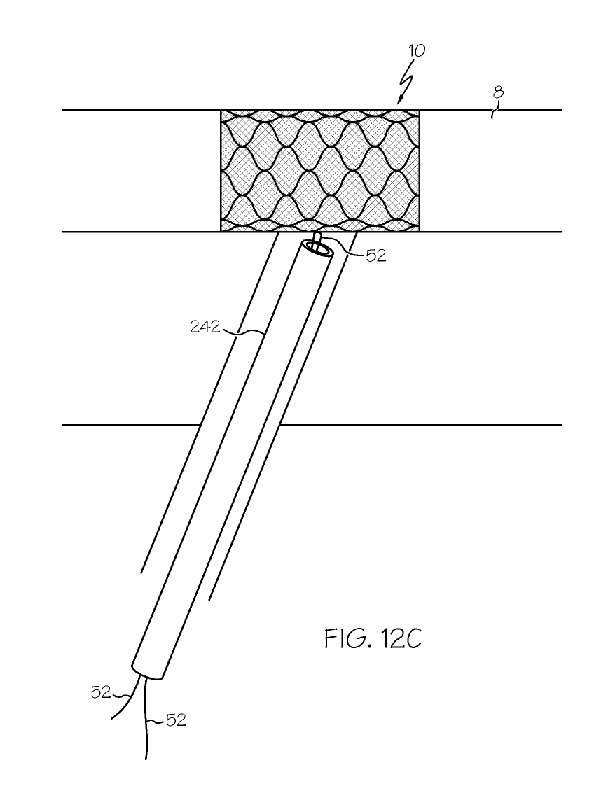

After the stent 12 has been properly positioned, it still needs to be expanded. To facilitate expansion of the stent 12, a free end or ends 148 of the string 52 is/are again pulled to release the string 52 from the stent 12 and permit the stent 12 to expand within the body lumen 8. As the string 52 is pulled, it unwraps from the stent assembly 10. In some embodiments, the string 52 is unwrapped from the ends of the stent 12 inward to prevent the string 52 from being trapped between the expanding stent 12 and the artery wall. Thus, the stent ends begin to expand before the remainder of the stent, with the middle of the stent expanding last, thereby permitting the strings to be easily removed from the lumen 8. In addition, in some embodiments, the portion(s) of the string 52 that are disposed longitudinally along the length of the stent (longitudinal string portions 78, shown in FIG. 12B) overlie the portions of the string 52 that are disposed circumferentially around the circumference of the stent or stent-graft (circumferential string portions 80). This string configuration aids in release of the string 52 from the stent 12.

In some embodiments, the stent 12 can be further aided in expansion by blood pressure exerted on the covering 14, thereby forcing the covering 14 up against the perforation 44 so the covering 14 traverses the perforation 44 and prevents blood from flowing outside the body lumen 8, as shown in FIG. 11C. The string 52 is pulled through the introducer sheath 41 and the introducer sheath 41 is removed from the patient.

With regard to the stent assembly 10 having a knit pattern 160, after the stent assembly 10 is positioned within the body lumen the strings 152 are pulled to release the strings 152 from the stent assembly and allow the stent to expand. Thus, the free end or ends 148 are pulled and the loops of the knit pattern become untied, similar to a chain stitch, crochet stitch, or feed bag stitch.

Turning now to FIGS. 12A-C, a stent assembly 10 is shown as hereinbefore described. As shown in FIGS. 12A-C, however, the stent assembly is inserted by way of a hollow pushrod 242. The string 52 is disposed through the hollow pushrod 252 and a portion thereof encircles at least a portion of the stent 12 and/or covering, as previously described. As shown in FIG. 12A, the stent or stent-graft is loaded into the introducer sheath 41 in a half-folded configuration. The hollow pushrod 242 is then used to push the stent or stent-graft into the artery or other body lumen 8. To deploy the stent or stent-graft, the string 52 is released from the stent or stent-graft. The string 52 is then removed from the artery or lumen 8 through the hollow center of the hollow pushrod 242, as shown in FIG. 12C. The stent can comprise a length to diameter ratio of about 1:1 to 2:1.

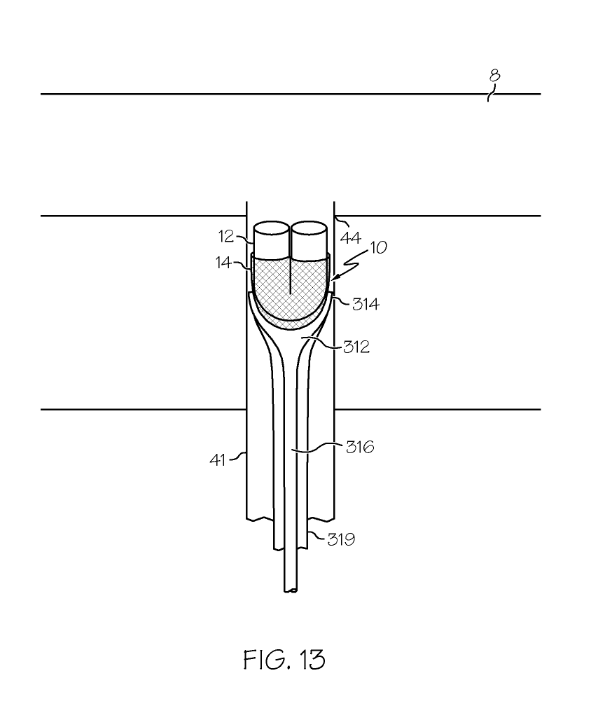

Turning to FIG. 13, stent assembly 10 is shown in an unexpanded and folded configuration. The stent assembly 10 comprises a stent 12 and a covering 14. The stent assembly is configured to be inserted into a body lumen 8 with a stent gripper 312. As shown in FIG. 13, the stent 12 and covering 14 are folded in half.

The stent gripper 312 comprises a wide portion 314 and a narrow portion 316. The wide portion 314 is configured to engage the stent 12 and the covering 14, thereby firmly gripping the stent assembly 10 in an unexpanded configuration.

The stent assembly 10 and stent gripper 312 are shown within a pull sheath 319, and the pull sheath 319, stent gripper 312, and stent assembly 10 are all disposed within an introducer sheath 41. In some embodiments, the sheath is an angioplasty sheath.

At least a portion of the pull sheath 319 surrounds at least a portion of the stent gripper 312. In some embodiments, a portion of the pull sheath 319 is slideably engaged to a wide portion 314 of the stent gripper 312. In this way, the pull sheath can be slid back away from the stent 12, permitting the wide portion 314 to expand and release the stent 12.

In some embodiments, the stent gripper can comprise an endoscopic biopsy forceps jaw, for example as shown and described in U.S. Pat. No. RE39,415, which is herein incorporated by reference. In some embodiments, the stent gripper can comprise a subcutaneous stent retrieval device.

To insert the stent assembly 10 within a body lumen 8, the stent gripper 312 and the pull sheath 319 are pushed through the introducer sheath 41 and into the lumen 8, so that the covering 14 will face the perforation 44 upon expansion. The pull sheath 319 is then pulled, permitting wide portion 314 of the stent gripper 312 to expand and release the stent assembly 10. The stent gripper 312 and the pull sheath 319 are then removed from the introducer sheath 41. The stent assembly 10 expands to an expanded configuration and the introducer sheath 41 is removed from the patient. As the stent assembly 10 expands to an expanded configuration, the covering 14 covers the perforation 44, thereby preventing blood or other bodily fluid from escaping from the body lumen 8.

As described above, the covering 14 comprises a self-sealing material 40, which permits subsequent re-entry into the stent assembly 10.

The stent gripper 312 is also capable of inserting a stent without a covering. Thus, the wide portion 314 is configured to engage a stent or portion thereof in a manner similar to that discussed above.

Although shown with only one fold in FIG. 13, in some embodiments, the stent assembly 10 can comprise a plurality of folds, for example between 4 and 10 folds, thereby permitting the stent to contract into a smaller unexpanded configuration.

Any of the stent assemblies disclosed herein can comprise biodegradable and/or bio-absorbable materials. For example, the covering 14 can be biodegradable, the stent 12 can be biodegradable, or both the sent 12 and the covering 14 can be biodegradable. In addition, any of the stent assemblies can comprise biodegradable, self-expanding materials, for example PLA, PGA, or PLGA.

In some embodiments, a stent can be implanted in accordance with the above description, without a covering. Thus, a stent can be implanted with a stent gripper or string(s) as discussed above. In this way, the stent gripper or string(s) can contact the stent, or a portion thereof, prior to or during implantation.

In some embodiments, the stent, stent assembly, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque. For example, the stent framework can comprise radiopaque struts.

In some embodiments, the stent or stent assembly is short enough to permit pannus ingrowth from both ends. Thus, in some embodiments, a microporous covering is unnecessary and a nonporous elastomer can be used. In some embodiments, for example where ingrowth along the length of the stent assembly is preferred, a covering containing micro-structures or pores to facilitate ingrowth may be used.

Description of some exemplary embodiments is contained in the following numbered paragraphs:

1. A stent assembly comprising:

a stent having an unexpanded configuration and an expanded configuration; and

a first string having a portion thereof formed in a repeating knit pattern; the repeating knit pattern portion of the first string encircling at least a portion of the stent in the unexpanded configuration.

2. The stent assembly of claim 1, wherein in the expanded configuration, the first string is removed from the stent.
3. The stent assembly of claim 1 further comprising a second string, the second string having a portion thereof formed in a repeating knit pattern; the repeating knit pattern portion of the second string encircling at least a portion of the stent in the unexpanded configuration;

wherein, when the stent is in the expanded configuration, the second string is removed from the stent.

4. The stent assembly of paragraph 1, wherein the first string comprises a first string section, a second string section, a third string section, a fourth string section, a fifth string section, and a sixth string section, the second string section and the third string section defining a first head end, the fifth string section and the sixth string section defining a second head end, the first head end comprising a first loop, the second head end comprising a second loop, the first loop disposed through the second loop.
5. The stent assembly of paragraph 1, wherein the repeating knit pattern comprises a pattern of first loops and second loops, each first loop facing a first direction and each second loop facing a second direction different from the first direction, wherein each first loop is adjacent to two second loops and each second loop is adjacent to two first loops.
6. The stent assembly of paragraph 1 further comprising at least one covering; the stent comprising a stent outer surface, the at least one covering immediately adjacent to at least a portion of the stent outer surface.
7. The stent assembly of paragraph 6, wherein the stent and the at least one covering are bio-degradable.
8. The stent assembly of paragraph 6, wherein the knit pattern portion of the first string encircles at least a portion of the at least one covering.
9. The stent assembly of paragraph 6, wherein the at least one covering comprises a self-sealing material.
10. The stent assembly of paragraph 6, wherein the self-sealing material comprises an elastomeric material.
11. The stent assembly of paragraph 6, wherein the at least one covering comprises a first covering and a second covering and the stent comprises a stent inner surface, wherein the first covering is immediately adjacent to at least a portion of the stent inner surface and the second covering is immediately adjacent to at least a portion of the stent outer surface.
11. The stent assembly of paragraph 11, wherein the first covering is thermally bonded to the second covering.
12. The stent assembly of paragraph 11, wherein the first covering is adhesively bonded to the second covering.
13. A stent assembly comprising:

a stent having an unexpanded configuration and an expanded configuration; and

a string having a portion thereof formed in a repeating serpentine path; the repeating serpentine path portion of the string encircling at least a portion of the stent in the unexpanded configuration.

14. The stent assembly of paragraph 14, wherein, in the expanded configuration, the string is removed from the stent.
15. The stent assembly of paragraph 13 further comprising at least one covering; the stent comprising a stent outer surface, the at least one covering immediately adjacent to at least a portion of the stent outer surface.
16. The stent assembly of paragraph 15, wherein the stent and the at least one covering are bio-degradable.
17 The stent assembly of paragraph 15, wherein the repeating serpentine path portion of the string encircles at least a portion of the at least one covering.
18. The stent assembly of paragraph 13, wherein the repeating serpentine path portion of the string is adhered to the stent assembly with an adhering material.
19. The stent assembly of paragraph 18, wherein the adhering material is selected from the group consisting of: sucrose, maltose, dextrose, and combinations thereof.
20. The stent assembly of paragraph 15, wherein the repeating serpentine path portion of the string encircles at least a portion of the at least one covering and is adhered thereto.
21. A stent assembly comprising:

a folded stent;

a covering encircling at least a portion of the folded stent;

a stent gripper; and

a pull sheath slidably engaged to at least a portion of the stent gripper;

the stent gripper releasably engaged to at least a portion of the stent covering.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

This completes the description of the preferred and alternate embodiments. Those skilled in the art may recognize other equivalents to the specific embodiment described herein which equivalents are intended to be encompassed by the claims attached hereto.

22. A method of sealing a puncture site in a body lumen comprising:

providing a stent having an outer surface and a covering over at least a portion of the outer surface;

providing a string encircling at least a portion of the stent;

placing the stent within the body lumen;

pulling on the string;

removing the string from the stent; and

deploying the stent such that the covering seals the puncture site.

What is claimed is:

1. A stent assembly comprising:
a stent having an unexpanded configuration and an expanded configuration; and
a first string having a portion thereof formed in a repeating knit pattern; the repeating knit pattern portion of the first string encircling at least a portion of the stent in the unexpanded configuration.

2. The stent assembly of claim 1, wherein in the expanded configuration, the first string is removed from the stent.

3. The stent assembly of claim 1 further comprising a second string, the second string having a portion thereof formed in a repeating knit pattern; the repeating knit pattern portion of the second string encircling at least a portion of the stent in the unexpanded configuration;
wherein, when the stent is in the expanded configuration, the second string is removed from the stent.

4. The stent assembly of claim 1, wherein the first string comprises a first string section, a second string section, a third string section, a fourth string section, a fifth string section, and a sixth string section, the second string section and the third string section defining a first head end, the fifth string section and the sixth string section defining a second head end, the first head end comprising a first loop, the second head end comprising a second loop, the first loop disposed through the second loop.

5. The stent assembly of claim 1, wherein the repeating knit pattern comprises a pattern of first loops and second loops, each first loop facing a first direction and each second loop facing a second direction different from the first direction, wherein each first loop is adjacent to two second loops and each second loop is adjacent to two first loops.

6. The stent assembly of claim 1 further comprising at least one covering; the stent comprising a stent outer surface, the at least one covering immediately adjacent to at least a portion of the stent outer surface.

7. The stent assembly of claim 6, wherein the stent and the at least one covering are bio-degradable.

8. The stent assembly of claim 6, wherein the knit pattern portion of the first string encircles at least a portion of the at least one covering.

9. The stent assembly of claim 6, wherein the at least one covering comprises a self-sealing material.

10. The stent assembly of claim 6, wherein the self-sealing material comprises an elastomeric material.

11. A stent assembly comprising:
a stent having an unexpanded configuration and an expanded configuration; and
a string having a portion thereof formed in a repeating serpentine path; the repeating serpentine path portion of the string encircling at least a portion of the stent in the unexpanded configuration.

12. The stent assembly of claim 11, wherein, in the expanded configuration, the string is removed from the stent.

13. The stent assembly of claim 13 further comprising at least one covering; the stent comprising a stent outer surface, the at least one covering immediately adjacent to at least a portion of the stent outer surface.

14. The stent assembly of claim 13, wherein the stent and the at least one covering are bio-degradable.

15. The stent assembly of claim 13, wherein the repeating serpentine path portion of the string encircles at least a portion of the at least one covering and is adhered thereto.
